(19) United States
(12) Patent Application Publication
(10) Pub. No.: US 2010/0266704 A1
(43) Pub. Date: Oct. 21, 2010

(54) OCTREOTIDE DEPOT FORMULATION WITH CONSTANTLY HIGH EXPOSURE LEVELS

(75) Inventors: Markus Ahlheim, Staufen (DE); Holger Petersen, Eimeldingen (DE)

Correspondence Address: NOVARTIS;CORPORATE INTELLECTUAL PROPERTY, ONE HEALTH PLAZA 101/2, EAST HANOVER, NJ, 07936-1080, US

(73) Assignee: NOVARTIS AG, Basel (CH)

(21) Appl. No.: 12/786,028

(22) Filed: May 24, 2010

Related U.S. Application Data
(63) Continuation-in-part of application No. 12/635,761, filed on Dec. 11, 2009, now abandoned.

(30) Foreign Application Priority Data
Dec. 15, 2008 (EP) 08171712.6

(51) Int. Cl. A61K 38/12; A61K 9/14

(52) U.S. Cl. 424/490; 514/21.1; 424/501

(57) ABSTRACT

The present invention relates to sustained release formulations comprising as active ingredient octreotide or a pharmaceutically-acceptable salt thereof and two different linear polylactide-co-glycolide polymers (PLGAs).

The present invention relates to sustained release formulations comprising as active ingredient octreotide or a pharmaceutically-acceptable salt thereof and certain linear polylactide-co-glycolide polymers (PLGAs).

The pharmaceutical compositions according to the present invention are indicated for inter alia long-term maintenance therapy in acromegalic patients, and treatment of severe diarrhea and flushing associated with malignant carcinoid tumors and vasoactive intestinal peptide tumors (vipoma tumors).

Peptide drugs are usually administered systemically, e.g. parenterally. However, parenteral administration may be painful and cause discomfort, especially for repeated daily administrations. In order to minimize the number of injections to a patient, the drug substance is advantageously administered as a depot formulation. A common drawback with injectable depot formulations is the fluctuation in plasma levels such as high peak levels together with plasma levels close to zero during the entire release period.

The present invention now provides an improved depot formulation providing constantly high exposure level. Furthermore, the depot formulation of the present invention reach the exposure level rapidly, i.e. have only a short or no lag phase. The depot formulations of the present invention comprise as active ingredient (drug substance) octreotide or a pharmaceutically-acceptable salt thereof. Octreotide is a somatostatin analog having the following formula:

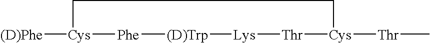

The active ingredient may be in the form of a pharmaceutically acceptable salt of octreotide, such as an acid addition salt with e.g. inorganic acid, polymeric acid or organic acid, for example with hydrochloric acid, acetic acid, lactic acid, citric acid, fumaric acid, malonic acid, maleic acid, tartaric acid, aspartic acid, benzoic acid, succinic acid or pamoic (embonic) acid.

Acid addition salts may exist as mono- or divalent salts, e.g. depending whether 1 or 2 acid equivalents are added. Preferred is the pamoate monosalt of octreotide.

To adequately control the hGH and IGF-1 levels of acromegaly patients typically a constant octreotide plasma level of as high as at least 1.5 ng/ml, 1.8 ng/ml or 2 ng/ml is required (therapeutic target plasma concentration). Developing a PLGA depot formulation which can constantly achieve these high plasma levels over an extended period of time has been proven very challenging. Sustained release formulations comprising as active ingredient octreotide or a pharmaceutically acceptable salt thereof and polylactide-co-glycolide polymers (PLGAs) have been described, for instance, in GB2265311, WO2007/071395 or WO2009/095450. However, the prior art formulations show either long phases of low levels (“lag phases”) as Batch 1-2 described in FIG. 1 and/or in between of the diffusion controlled release and the erosion controlled release a “valley” as Batch 1-2 and 1-3 described in FIG. 1. Furthermore, so far, none of the described octreotide depot formulations have been able to meet therapeutic target plasma level with a dosage of 12 mg/kg body weight in rabbits (Male New Zealand White rabbits (Hsdlf:NZW), ˜3 kg±20% at arrival (Harlan Netherlands)) over an extended time of more than 50 days.

It has now surprisingly found in accordance with the present invention that octreotide depot formulations comprising two different linear PLGA polymers having a molar ratio of lactide:glycolide comonomer (L:G) from 85:15 to 65:35, and at least one polymer has a low inherent viscosities, e.g. an inherent viscosity of 0.6 dl/g or below provide a favorable release profile, in particular with respect to constant high exposure level, short lag phase and/or the reduction or absence of a (sub-therapeutic) trough. The formulations of the present invention have been found to be able to provide sustained high octreotide plasma levels of at least 1.5 ng/ml, 1.8 ng/ml or 2 ng/mL for extended period of time such as e.g. at least 1 month, 6 weeks or 50 days. The favorable release profile over an extended time is therefore particularly suitable for a sustained release formulation which can be applied over a longer time than currently marketed sustained release formulation of octreotide, also know as Sandostatin® LAR®, which is administered every 28 days. Furthermore, the favorable PK profile of the octreotide depot formulations according to present invention are have been found to be particularly suitable for a one month or 6 weeks subcutaneous octreotide depot formulation.

The present invention provides an octreotide depot formulation composed of a mixture or a blend of two different PLGA polymers having both a molar L:G ratio from 85:15 to 65:35, preferably 85:15 or 75:25, more preferably 75:25. In one preferred embodiment, both polymers have a molar L:G ratio of 75:25 but different inherent viscosities. In one embodiment, at least one of the polymers has a low inherent viscosity, e.g. an inherent viscosity below 0.6 dl/g, 0.55 dl/g, 0.5 dl/g, 0.45, 0.4 dl/g, 0.35 dl/g, 0.3 dl/g, 0.25 dl/g or 0.2 dl/g. In a preferred embodiment, the two PLGA polymers have different inherent viscosity, e.g. an inherent viscosity of 0.6 and 0.4 dl/g, 0.6 dl/g and 0.2 dl/g, 0.4 dl/g and 0.2 dl/g. In one preferred embodiment, at least one of the polymers has an inherent viscosity of below 0.5 dl/g or 0.4 dl/g, e.g. 0.2 dl/g. Preferably, the two polymers differ in inherent viscosity between 0.2 dl/g to 0.4 dl/g, preferably 0.2 dl/g. As understood by the skilled person, an inherent viscosity and L:G molar ratio in the context of the polymers according to the present invention are an average over a certain range as e.g. indicated in the specifications of the manufacturer. In another preferred embodiment, the different polymers preferably have different end groups, e.g. an ester and a carboxy end group.

Suitable polymers are commonly known but not limited to those commercially available as RESOMER® by Boehringer Ingelheim Pharma GmbH & Co. KG, Ingelheim, Germany, LACTEL® by Absorbable Polymers International (API), Pelham, Ala., USA, MEDISORB® by Alkermes, Inc., Cambridge, Mass., USA, PURASORB® by PURAC biochem BV, Gorinchem, The Netherlands. Examples of suitable polymers are listed in Table 1.

TABLE 1
Examples of suitable polymers

| | | | Inherent | Producer |
|---|---|---|---|---|
| No | Product name | Polymer | viscosity [dL/g] | Supplier |
| 1 | Resomer ® R 202 H | Linear Poly(D,L-lactide) | 0.16-0.24 ¹⁾ | Boehringer |
| | | free carboxylic acid end group | | |
| 2 | Resomer ® R 202 S | Linear Poly(D,L-lactide) | 0.16-0.24 ¹⁾ | Boehringer |
| 3 | Resomer ® R 203 S | Linear Poly(D,L-lactide) | 0.25-0.35 ¹⁾ | Boehringer |
| 4 | Resomer ® RG 752 H | Linear Poly(D,L-lactide-co- | 0.14-0.22 ¹⁾ | Boehringer |
| | | glycolide) 75:25 | | |
| | | free carboxylic acid end group | | |
| 5 | Resomer ® RG 752 S | Linear Poly(D,L-lactide-co- | 0.16-0.24 ¹⁾ | Boehringer |
| | | glycolide) 75:25 | | |
| 6 | Resomer ® RG 753 S | Linear Poly(D,L-lactide-co- | 0.32-0.44 ¹⁾ | Boehringer |
| | | glycolide) 75:25 | | |
| 7 | Lactel ® 100D020A | Linear Poly(D,L-lactide) | 0.15-0.25 ²⁾ | API/Durect |
| | | free carboxylic acid end group | | |
| 8 | Lactel ® 100D040A | Linear Poly(D,L-lactide) | 0.26-0.54 ²⁾ | API/Durect |
| | | free carboxylic acid end group | | |
| 9 | Lactel ® 100D040 | Linear Poly(D,L-lactide) | 0.26-0.54 ²⁾ | API/Durect |
| 10 | Lactel ® 100D065 | Linear Poly(D,L-lactide) | 0.55-0.75 ²⁾ | API/Durect |
| 11 | Lactel ® 85DG040 | Linear Poly(D,L-lactide-co- | 0.26-0.54 ²⁾ | API/Durect |
| | | glycolide) 85:15 | | |
| 12 | Lactel ® 85DG065 | Linear Poly(D,L-lactide-co- | 0.55-0.75 ²⁾ | API/Durect |
| | | glycolide) 85:15 | | |
| 13 | Lactel ® 75DG065 | Linear Poly(D,L-lactide-co- | 0.55-0.75 ²⁾ | API/Durect |
| | | glycolide) 75:25 | | |
| 14 | Lactel ® 65DG065 | Linear Poly(D,L-lactide-co- | 0.55-0.75 ³⁾ | API/Durect |
| | | glycolide) 65:35 | | |
| 15 | Lactel ® 50DG065 | Linear Poly(D,L-lactide-co- | 0.55-0.75 ³⁾ | API/Durect |
| | | glycolide) 50:50 | | |
| 16 | Medisorb ® | Linear Poly(D,L-lactide) | 0.66-0.80 | Alkermes |
| | 100 DL HIGH IV | | | |
| 17 | Medisorb ® | Linear Poly(D,L-lactide) | 0.50-0.65 | Alkermes |
| | 100 DL LOW IV | | | |
| 18 | Medisorb ® | Linear Poly(D,L-lactide-co- | 0.66-0.80 | Alkermes |
| | 8515 DL HIGH IV | glycolide) 85:15 | | |
| 19 | Medisorb ® | Linear Poly(D,L-lactide-co- | 0.50-0.65 | Alkermes |
| | 8515 DL LOW IV | glycolide)85:15 | | |
| 20 | Medisorb ® | Linear Poly(D,L-lactide-co- | 0.66-0.80 | Alkermes |
| | 7525 DL HIGH IV | glycolide) 75:25 | | |
| 21 | Medisorb ® | Linear Poly(D,L-lactide-co- | 0.50-0.65 | Alkermes |
| | 7525 DL LOW IV | glycolide) 75:25 | | |
| 22 | Medisorb ® | Linear Poly(D,L-lactide-co- | 0.66-0.80 | Alkermes |
| | 6535 DL HIGH IV | glycolide) 65:35 | | |
| 23 | Medisorb ® | Linear Poly(D,L-lactide-co- | 0.50-0.65 | Alkermes |
| | 6535 DL LOW IV | glycolide) 65:35 | | |
| 24 | Medisorb ® | Linear Poly(D,L-lactide-co- | 0.66-0.80 | Alkermes |
| | 5050 DL HIGH IV | glycolide) 50:50 | | |
| 25 | Medisorb ® | Linear Poly(D,L-lactide-co- | 0.50-0.65 | Alkermes |
| | 5050 DL LOW IV | glycolide) 50:50 | | |

¹⁾ IV has been determined in chloroform at a concentration of 0.1% at 25° C.
²⁾ IV has been determined in chloroform at a concentration of 0.5 g/dL at 30° C.
³⁾ IV has been determined in Hexafluoroisopropanol at a concentration of 0.5 g/dL at 30° C.

In one embodiment the present invention provides extended release octreotide depot formulations which show constantly a high exposure for at least 50 days, preferably at least about 2 months, in rabbits after i.m. injection. Furthermore, the extended release depot formulations of the present invention show a short lag phase until the therapeutic target level is reached. For a single injection, a typical lag phase between the initial burst and reaching the therapeutic target plasma concentration of the extended release depot formulations of the present invention is shorter than 12 days, e.g. between 4 to 12 days or 6 to 10 days. In a preferred embodiment, the present invention provides a microparticle extended release formulation comprising octreotide or an octreotide salt, e.g. octreotide pamoate, exhibiting a sustained high octreotide plasma levels of at least 1.5 ng/ml, 1.8 ng/ml or 2 ng/mL for a period of at least 50 days. Such a formulation can for instance be administered in a suitable vehicle intramuscularly (i.m.) e.g. via deep i.m. injection. Such injections are typically administered by experienced clinical professionals (experienced physicians or nurses).

In another embodiment, the present invention provides a high exposure depot formulation comprising octreotide or an octreotide salt, e.g. octreotide pamoate, for subcutaneous administration. Such a formulation can for instance be administered in a suitable vehicle by an experienced clinical professional or by the patient (self administration). The s.c. depot formulations of the present invention typically show immediate action, i.e. therapeutic plasma concentrations are achieved in short time (e.g. after 2, 3, 4, 5, 6 or 7 days, typically after 5,6 or 7 days) after s.c. injection. Furthermore, the s.c. depot formulations typically show constantly high exposure levels (i.e. do not have a trough with sub-therapeutic plasma level) over about 1 month or longer, e.g. up to 6 weeks. Typical drug loads of such formulations are e.g. 10 to 25%, preferably 15% to 25%, e.g. about 20% of the free octreotide. The surprisingly high bioavailability and the high drug load of the octreotide depot formulation according to the present invention enables a lower dosage strength for a octreotide 1 month s.c. depot formulation as compared with the currently marketed sustained release formulation of octreotide, Sandostatin® LAR® which needs to be administered deep i.m. For instance, a dose of 20 mg octreotide administered subcutaneously with a high exposure depot formulation of the present invention may be equivalent to a dosage strength of 30 mg of Sandostatin® LAR® administered intramuscularly. Previously described octreotide depot formulations are not suitable for a subcutaneous injection due to the high injection volume (e.g. 2.5 ml for Sandostatin® LAR®). The present invention now provides octreotide depot formulations that can be administered in smaller injection volumes due to high bioavailability and high drug load, e.g. in an injection volume of 0.75 to 1.5 ml, e.g. in 1 ml. The octreotide depot formulations of the present invention can be injected using a smaller needle, e.g. 25 G×⅝″.

The two different PLGA polymers can be mixed or blended in a % wt ratio of 95:5 to 50:50, preferably 85:15 to 50:50 or 80:20 to 60:40, e.g. 90:10, 85:15, 80:20, 75:25, 70:30, 65:35, 60:40, 55:45 or 50:50% wt, preferably 80:20, 70:30 or 60:40% wt, more preferably 70:30% wt. In a preferred embodiment, the polymer with the higher inherent viscosity has a higher % wt than the polymer with the lower inherent viscosity. In another preferred embodiment, the polymer with the higher inherent viscosity has an ester end-group.

The octreotide depot formulations in accordance with the present invention may comprise further polymers, e.g. polymers such as other linear or star shaped PLGA polymers, or PLG or PLA polymers, as long as the favorable PK properties of the present invention are retained.

In one embodiment, the present invention provides methods of treatment for diseases that respond to treatment with somatostatin analogues, e.g. long-term maintenance therapy in acromegalic patients, and treatment of severe diarrhea and flushing associated with malignant carcinoid tumors and vasoactive intestinal peptide tumors (vipoma tumors), using a depot formulation according to the present invention. The depot formulation can e.g. be administered subcutaneously (e.g. using a 25 G×⅝″ or 23 G×1″ needle) in a dosage strength of about 5 to 25 mg, e.g. 5 mg, 10 mg, 15 mg or 20 mg; or e.g. 7 mg, 14 mg or 21 mg.

In another embodiment, present invention provides depot formulations comprising two different linear polylactide-co-glycolide polymers (PLGAs) having a molar L:G ratio of 85:15 to 65:35 and as active ingredient octreotide, or a pharmaceutically acceptable salt thereof, e.g. octreotide pamoate, and having two different inherent viscosities which are 0.6 dl/g or less (e.g. 0.6 dl/g or 0.4 dl/g or 0.2 dl/g), for use in the treatment of a disease that responds to the treatment with somatostatin analogues, wherein said depot formulation is to be administered subcutaneously about once about every month (e.g. once every 25, 26, 27, 28, 29, 30, 31, 32, 33, 34 or 35 days) or about every six weeks.

The particle size distribution of the drug substance influences the release profile of the drug from the depot form. The drug substance which is used to prepare the depot formulation is crystalline or in the form of an amorphous powder. Preferred is an amorphous powder which has a particle of a size of about 0.1 microns to about 15 microns (99%>0.1 microns, 99%<15 microns), preferably from 1 to less than about 10 microns (90%>1 microns, 90%<10 microns). The drug substance preferentially undergoes a micronization process to present the required particle size distribution.

The present invention further provides a sustained release pharmaceutical composition (depot) comprising as active ingredient octreotide or a pharmaceutically-acceptable salt thereof incorporated in a poly(lactide-co-glycolide)s (PLGAs) matrix, for instance in form of microparticles, implants or semisolid formulations.

The extended release pharmaceutical composition according to the present invention, e.g. for i.m. administration, allows a sustained release of the active ingredient in a patient in need (preferably a human) over a period of at least 20, 28, 30, 45 days, at least 50 days, at least 60 days, at least 75 days or at least 90 days. During the release of the active ingredient the plasma levels of octreotide are within the therapeutic range for 20 to 70 days.

It is understood that the exact dose of octreotide will depend on a number of factors, including the condition to be treated, the severity of the condition to be treated, the weight of the subject and the duration of therapy. The favorable release profile of the present invention allows for longer administration intervals of the pharmaceutical compositions of the present invention as compared to the prior art formulations. So far, no octreotide depot formulation with longer dosing intervals than every 28 days have been approved for therapy. The depot formulations of the present invention are now, due to their favorable release profile, suitable for administration once every 1 month up to once every 2 months (e.g. every 4 weeks up to every 8 weeks).

Fluctuations in plasma levels can be significantly reduced by using a suitable combination of two different linear PLGAs in the pharmaceutical composition according to the present invention.

In particularly preferred embodiments, both PLGAs have a L:G molar ratio of 75:25 or both PLGAs have a L:G ratio of 85:15 or one PLGA has a L:G ratio of 85:15 and one has a L:G ratio of 75:25. Typical examples of such preferred embodiments include two PLGAs having a L:G molar ratio of 75:25 with inherent viscosities of 0.6 dl/g and 0.4 dl/g, or 0.6 dl/g and 0.2 dl/g, or preferably, 0.4 dl/g and 0.2 dl/g. Further typical example include two PLGAs having a L:G molar ratio of 85:15 with inherent viscosities of 0.6 dl/g and 0.4 dl/g, or 0.6 dl/g and 0.2 dl/g, or preferably, 0.4 dl/g and 0.2 dl/g. In one embodiment, the two polymers have the same end-group, e.g. an acid or ester group. In a preferred embodiment, the polymers have different end-groups, e.g. an acid end-group on the polymer with the higher viscosity and an ester end-group at the polymer with the lower viscosity or an ester end-group on the polymer with the higher viscosity and an acid end-group at the polymer with the lower viscosity.

The PLGAs according to the present invention have a molecular weight (Mw) ranging from 1,000 to 500,000 Da, preferably from 5,000 to 100,000 Da. The architecture of the polymers is linear.

The inherent viscosity (IV) of the PLGAs according to the present invention is below 0.9 dl/g in CHCl₃, preferentially below 0.8 dl/g, preferably below 0.6 dl/g, more preferably between 0.1 dl/g to 0.5 dl/g in CHCl₃. The inherent viscosities can be measured by the conventional methods of flow time measurement, as described for example in “Pharmacopoée Européenne”, 1997, pages 17-18 (capillary tube method). Unless stated otherwise, these viscosities have been measured at 25° C. at a concentration of 0.1% in CHCl₃.

End groups of the PLGAs according to the present invention can be but are not limited to Hydroxy, carboxy, ester or the like.

The drug substance content of the depot formulation (the loading) is in a range of 1% to 30%, preferred 10% to 25%, more preferred 15% to 20%. The loading is defined as the weight ratio of drug substance as free base to the total mass of the PLGA formulation.

Suitable polymers are commonly known but not limited to those commercially available as RESOMER® by Boehringer Ingelheim Pharma GmbH & Co. KG, Ingelheim, Germany, LACTEL® by Durect Corp., Pelham, Ala., USA, MEDISORB® by Lakeshore, Inc., Cambridge, Mass., USA, PURASORB® by PURAC biochem BV, Gorinchem, The Netherlands. Particularly preferred polymers of the present invention are Resomer® RG 752H and Resomer® RG 753 S.

The pharmaceutical composition according to the present invention can be manufactured aseptically or non-aseptically and sterilized terminally by gamma irradiation. Preferred is terminal sterilization by gamma irradiation, resulting in a product with the highest sterility assurance possible.

The pharmaceutical composition according to the present invention may also contain one or more pharmaceutical excipients modulating the release behavior in an amount of 0.1% to 50%. Examples of such agents are: Polyvinyl alcohol, Polyvinyl pyrrolidone, carboxymethyl cellulose sodium (CMC—Na), dextrin, polyethylene glycol, suitable surfactants such as poloxamers, also known as poly(oxyethylene-block-oxypropylene), Poly(oxyethylene)-sorbitan-fatty acid esters known and commercially available under the trade name TWEEN® (e.g. Tween 20, Tween 40, Tween 60, Tween 80, Tween 65 Tween 85, Tween 21, Tween 61, Tween 81), Sorbitan fatty acid esters e.g. of the type known and commercially available under the trade name SPAN, Lecithins, inorganic salts such as zinc carbonate, magnesium hydroxide, magnesium carbonate, or protamine, e.g. human protamine or salmon protamine, or natural or synthetic polymers bearing amine-residues such as polylysine.

The pharmaceutical composition according to the present invention can be a depot mixture or a polymer blend of different polymers in terms of compositions, molecular weight and/or polymer architectures. A polymer blend is defined herein as a solid solution or suspension of two different linear polymers in one implant or microparticle. A mixture of depots in contrast is defined herein as a mixture of two depots like implants or microparticles or semisolid formulations of different composition with one or more PLGAs in each depot. Preferred is a pharmaceutical composition wherein the two PLGAs are present as polymer blend.

The pharmaceutical composition according to the present invention can be in the form of implants, semisolids (gels), liquid solutions or suspensions which solidify in situ once they are injected or microparticles. Preferred are microparticles. Preparation of microparticles comprising octreotide or a pharmaceutically-acceptable salt thereof is known and for instance disclosed in U.S. Pat. No. 5,445,832 or U.S. Pat. No. 5,538,739.

The following part of the invention is focused on polymer microparticles although the descriptions are applicable for implants, semisolids and liquids as well.

The microparticles according to the present invention may have a diameter from a few submicrons to a few millimeters, e.g. from about 0.01 microns to about 2 mm, e.g. from about 0.1 microns to about 500 microns. For pharmaceutical microparticles, diameters of at most about 250 microns, e.g. 10 to 200 microns, preferably 10 to 130 microns, more preferably 10 to 90 microns.

The microparticles according to the present invention may be mixed or coated with an anti-agglomerating agent or covered by a layer of an anti-agglomerating agent, e.g. in a prefilled syringe or vial. Suitable anti-agglomerating agents include, e.g. mannitol, glucose, dextrose, sucrose, sodium chloride, or water soluble polymers such as polyvinyl alcohol, polyvinyl pyrrolidone or polyethylene glycol, e.g. with the properties described above.

The manufacturing process for the depot formulation of the current invention is described in detail for microparticles:

The microparticles may be manufactured by several processes known in the art, e.g., coacervation or phase separation, spray drying, water-in-oil (W/O) or water-in-oil-in-water (W/O/W) or solids-in-oil-in-water (S/O/W) emulsion/suspension methods followed by solvent extraction or solvent evaporation. The emulsion/suspension method is a preferred process, which comprises the following steps:

(i) preparation of an internal organic phase comprising
(ia) dissolving the polymer or polymers in a suitable organic solvent or solvent mixture; optionally dissolving/dispersing suitable additives;
(ib) dissolving/suspending/emulsification of the drug substance in the polymer solution obtained in step (ia);
   (ii) preparation of an external aqueous phase containing stabilizers and optionally but preferably buffer salts;
   (iii) mixing the internal organic phase with the external aqueous phase e.g. with a device creating high shear forces, e.g. with a rotor-stator mixer (turbine) or static mixer, to form an emulsion; and
   (iv) hardening the microparticles by solvent evaporation or solvent extraction, washing the microparticles, e.g. with water, collecting and drying the microparticles, e.g. freeze-drying or drying under vacuum, and sieving the microparticles through 140 μm.

Suitable organic solvents for the polymers include e.g. ethyl acetate, acetone, THF, acetonitrile, or halogenated hydrocarbons, e.g. methylene chloride, chloroform or hexafluoroisopropanol.

Suitable examples of a stabilizer for step (iib) include Poly(vinylalcohol) (PVA), in an amount of 0.1 to 5%, Hydroxyethyl cellulose (HEC) and/or hydroxypropyl cellulose (HPC), in a total amount of 0.01 to 5%, Poly(vinyl pyrolidone), Gelatin, preferably porcine or fish gelatin.

The dry microparticles composition can be terminally sterilized by gamma irradiation (overkill sterilization), optionally in bulk or after filling in the final container resulting in the highest sterility assurance possible. Alternatively the bulk sterilized microparticles can be resuspended in a suitable vehicle and filled as a suspension into a suitable device such as double chamber syringe with subsequent freeze drying.

The pharmaceutical composition according to the present invention containing microparticles may also contain a vehicle to facilitate reconstitution.

Prior to administration, the microparticles are suspended in a suitable vehicle for injection. Preferably, said vehicle is water based containing pharmaceutical excipients such as mannitol, sodium chloride, glucose, dextrose, sucrose, or glycerins, non-ionic surfactants (e.g. poloxamers, poly(oxyethylene)-sorbitan-fatty acid esters, carboxymethyl cellulose sodium (CMC—Na), sorbitol, poly(vinylpyrrolidone), or aluminium monostearate in order to ensure isotonicity and to improve the wettability and sedimentation properties of the microparticles. The wetting and viscosity enhancing agents may be present in an amount of 0.01 to 1%; the isotonicity agents are added in a suitable amount to ensure an isotonic injectable suspension.

The invention further provides the use of a pharmaceutical composition according to the present invention for inter alia long-term maintenance therapy in acromegalic patients, and treatment of severe diarrhea and flushing associated with malignant carcinoid tumors and vasoactive intestinal peptide tumors (vipoma tumors).

The utility of the pharmaceutical compositions according to the present invention can be shown in standard clinical or animal studies.

The invention further provides a kit comprising the depot formulation in a vial, optionally equipped with a transfer set, together with a water-based vehicle in an ampoule, vial or prefilled syringe or as microparticles and vehicle separated in a double chamber syringe.

### BRIEF DESCRIPTION OF THE DRAWINGS

FIG. 1 shows examples 1-1, 1-2 and 1-3 (formulation variants C, B and A) in comparison. Octreotide serum conc. over time after 12 mg/kg dosage i.m. into rabbits. Mean and SD of 4 animals.

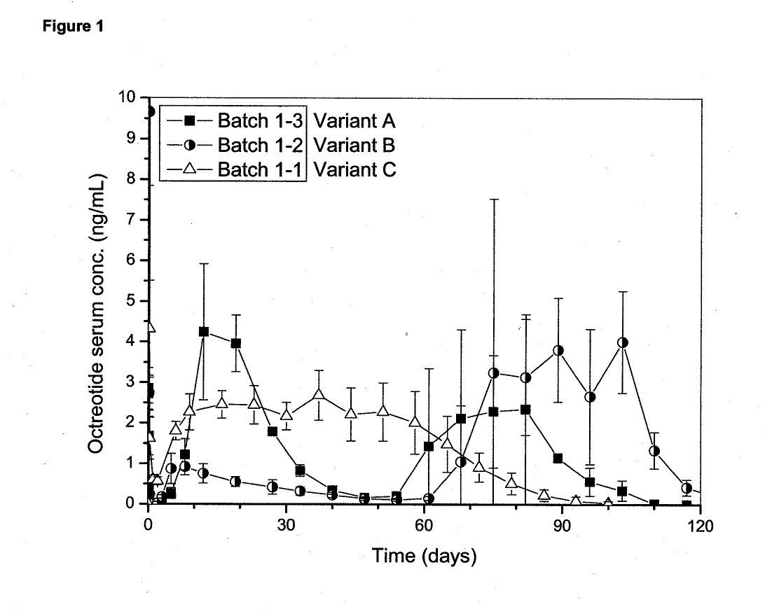

FIG. 2 shows examples 1-1,1-4, 1-5 and 1-6 (formulation variants C, C2, C3 and C4) in comparison. Octreotide serum conc. over time after 12 mg/kg dosage i.m. into rabbits. Mean and SD of 4 animals.

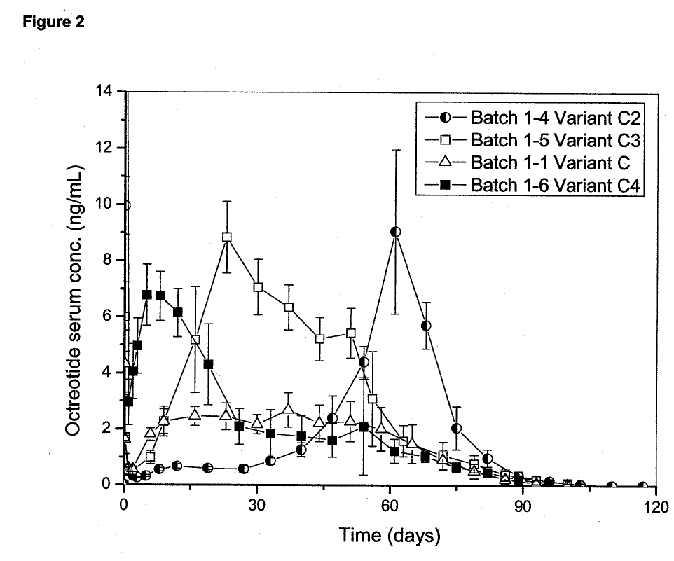

FIG. 3 shows examples 1-1, 1-7 and 1-8 (formulation variants C, C5 and D) in comparison. Octreotide serum conc. over time after 12 mg/kg dosage i.m. into rabbits. Mean and SD of 4 animals.

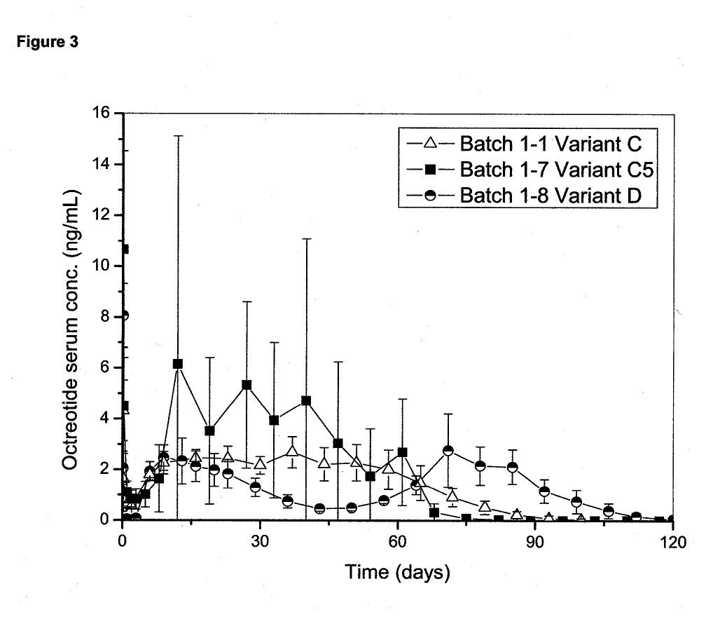

FIG. 4 shows example 1-1 (formulation variant C) after i.m. and s.c. injection in comparison. Octreotide serum conc. over time after 4 mg/kg dosage i.m. and s.c. and 12 mg/kg dosage i.m. into rabbits. Mean and SD of 4 animals.

### EXPERIMENTAL PART

The following examples are illustrative, but do not serve to limit the scope of the invention described herein. The examples are meant only to suggest a method of practicing the present invention.

### Example 1

### Microparticle Preparation

An appropriate amount of the PLGA polymers is dissolved in an appropriate amount of dichloromethane to give an appropriate polymer concentration as stated in column “PLGA conc.” in Table 2. An appropriate amount of drug substance is weight into a glass beaker and the polymer solution is poured over the drug substance so that the resulting microparticles have a drug load as stated in column “drug load”.

E.g. for microparticles with a drug load of 20% and a polymer concentration of 20% the numbers are as the following: 3.547 g of the PLGA polymers are dissolved into 17.7 ml dichloromethane to give a 20% (w/v) polymer solution. 1.453 g of octreotide pamoate with a free peptide content of 68.8% (corresponding to 1.00 g=20% octreotide free base) is weight into a glass beaker and the polymer solution is poured over the drug substance.

The suspension is homogenized with an Ultra-Turrax rotor-stator mixer with 20,000 rpm for 1 min under cooling with an ice/water mixture. This suspension is referred to as S/O suspension.

10.00 g of Polyvinylalcohol PVA 18-88, 3.62 g KH₂PO₄ and 15.14 g Na₂HPO₄ are dissolved in 2.00 L deionized water to form a 0.5% PVA 18-88 solution buffered to pH 7.4.

The S/O suspension is mixed with the 0.5% PVA18-88 solution by pumping the S/O suspension with the help of a flexible tube pump (Perpex, Viton tube) at a rate of 10 ml/min into a turbine and by pumping the aqueous solution with a gear pump (Ismatec MV-Z/B with pumping head P140) at a rate of 200 ml/min into the same turbine. The two solutions are mixed in the turbine as described in Table 2. The homogenized S/O/W emulsion is collected into a 2 L glass beaker which is prefilled with 200 ml of the buffered PVA solution.

The S/O/W emulsion is then heated up to 45° C. in 5 h. The temperature of 45° C. is hold for further 2 h min, before the batch is cooled to room temperature again. During this process escaping dichloromethane is removed by vacuum and the batch is stirred by a 4 blade-propeller-stirrer at 250 rpm.

As a result, microparticles are formed out of the S/O/W emulsion. The microparticles are collected by filtration (5 μm). They are washed 5 times with 200 ml water and dried for 36 h at 20° C. and 0.030 mbar. The dried microparticles are sieved through 140 μm and filled under nitrogen into glass vials. Prepared in that way, the microparticles are sterilized by gamma-irradiation with a dose of 30 kGy.

The particle size of the microparticles is measured by laser light diffraction. The microparticles are resuspended in white spirit using ultra sound. Table 2 gives the diameter ×90 (90% of all particles are smaller than this value) after 120 seconds of ultra sound treatment.

The assay of the microparticles is determined by HPLC after dissolving the microparticles with ultra sound in a 3:2 mixture of acetonitrile and methanol and further 1:1 dilution with a sodium acetate buffer (pH 4). The solution is cleared from residual particulate matter by centrifugation.

TABLE 2
Examples 1-1, 1-4, 1-5, 1-6 and 1-7: octreotide pamoate microparticles prepared
by blend of two linear PLGAs (75:25). Comparative examples 1-2, 1-3 and 1-8:
octreotide pamoate microparticles prepared by blend of two or three linear PLGAs.

| | Drug | PLGA | | | | | | | Turbine | | |
|---|---|---|---|---|---|---|---|---|---|---|---|
| Ex. | Load | conc. | | | | | | | speed | Particle size | Assay |
| Batch | (%) | (%) | A | B | C | D | E | F | (rpm) | x₉₀ (μm) | (%) |
| 1-1 | 20 | 20 | — | 30 | — | 70 | — | — | 2800 | 60 | 18.4 |
| Var C | | | | | | | | | | | |
| 1-2 | 20 | 20 | 33 | — | — | 34 | — | 33 | 3800 | 68.4 | 19.6 |
| Var B | | | | | | | | | | | |
| 1-3 | 20 | 20 | — | — | — | 50 | — | 50 | 4500 | 58.6 | 18.6 |
| Var A | | | | | | | | | | | |
| 1-4 | 20 | 20 | — | 10 | — | 90 | — | — | 3000 | 54.5 | 19.3 |
| Var C2 | | | | | | | | | | | |
| 1-5 | 20 | 20 | — | 20 | — | 80 | — | — | 2800 | 60.5 | 18.0 |
| Var C3 | | | | | | | | | | | |
| 1-6 | 20 | 20 | — | 30 | — | — | 70 | — | 2800 | 70.3 | 20.3 |
| Var C4 | | | | | | | | | | | |
| 1-7 | 20 | 20 | | 10 | 90 | | | | 2300 | 71 | 20.7 |
| Var C5 | | | | | | | | | | | |
| 1-8 | 20 | 20 | | 33 | | 34 | | 33 | 3500 | 62.8 | 17.4 |
| Var D | | | | | | | | | | | |

A: PLGA 65:35 ester 0.6 dL/g (%)
B: PLGA 75:25 acid 0.2 dL/g (%)
C: PLGA 75:25 ester 0.2 dL/g (%)
D: PLGA 75:25 ester 0.4 dL/g (%)
E: PLGA 75:25 ester 0.6 dL/g (%)
F: PLGA 85:15 ester 0.6 dL/g (%)

### Example 2

### Vehicle Compositions A to G

CMC—Na, Mannitol and Pluronic F68 in an amount as given in Table 3 are dissolved in about 15 ml hot deionized water of a temperature of about 90° C. under strong stirring with a magnetic stirrer. The resulting clear solution is cooled to 20° C. and filled up with deionized water to 20.0 ml.

TABLE 3
Suitable vehicles for the microparticles (Amounts given in g)

| | A | B | C | D | E | F | G |
|---|---|---|---|---|---|---|---|
| CMC-Na | 0 | 0 | 0.05 | 0.14 | 0.28 | 0.35 | 0.40 |
| Mannitol | 0 | 1.04 | 0.99 | 0.90 | 0.76 | 0.74 | 0.68 |
| Pluronic F68 | 0.04 | 0.04 | 0.04 | 0.04 | 0.04 | 0.04 | 0.04 |

### Example 3

### Microparticle Suspension

180 mg of microparticles of example 1-1,1-2, 1-3,1-4, 1-5,1-6, 1-7 or 1-8 are suspended in 1.0 ml of a vehicle of composition D (Table 3) in a 6 R vials. The suspensions are homogenized by shaking for about 30 seconds by hand. The reconstituted suspension may be injected without any issues using a 20 Gauge needle.

### Example 4

### Lyophilisation of the Microparticles

180 mg of microparticles of example 1-1,1-2, 1-3,1-4, 1-5,1-6, 1-7 or 1-8 are reconstituted in 1 ml of the vehicle composition F (Table 3), homogenized by stirring for 1 to 12 hours and then freeze-dried in a lyophilisator. Reconstitution of the lyophilized microparticles with 1 ml pure water (aqua ad injectabilia) resulted in fast and good wetting of the microparticles that may be injected without any issues using a 20 Gauge needle.

### Example 5

### Release Profile In Vivo (Rabbits)

Microparticles containing octreotide are suspended in 1 ml of a suitable aqueous vehicle and the resulting suspension is injected intramusculary (i.m.) into male New Zealand White rabbits in a dose of 12 mg/kg. For each dosage form (test group) 4 animals are used. After defined time periods (indicated in the table 4) plasma samples are taken and analyzed for octreotide concentration by radioimmunoassay (RIA).

TABLE 4
Plasma levels Example 1-1

| Time [days]/ | | | | | Mean or | |
|---|---|---|---|---|---|---|
| Subject No. | 473 | 474 | 476 | 480 | Range‡ | SD |
| 0 | 0.000 | 0.000 | 0.000 | 0.000 | 0.000 | 0.000 |
| 0.021 | 56.026 | 41.316 | 52.099 | 48.148 | 49.397 | 6.274 |
| 0.042 | 40.769 | 50.921 | 37.531 | 30.494 | 39.929 | 8.491 |
| 0.083 | 16.154 | 25.658 | 15.185 | 11.889 | 17.222 | 5.913 |
| 0.167 | 4.590 | 5.408 | 4.654 | 2.617 | 4.317 | 1.193 |
| 0.25 | 2.103 | 1.987 | 1.383 | 1.006 | 1.620 | 0.517 |
| 1 | 0.763 | 0.597 | 0.503 | 0.517 | 0.595 | 0.119 |
| 2 | 0.579 | 0.694 | 0.513 | 0.476 | 0.566 | 0.096 |
| 6 | 1.769 | 2.105 | 1.556 | 1.802 | 1.808 | 0.226 |
| 9 | 2.218 | 2.895 | 2.099 | 1.864 | 2.269 | 0.442 |
| 16 | 2.744 | 2.750 | 2.198 | 2.136 | 2.457 | 0.336 |
| 23 | 2.436 | 3.118 | 2.185 | 2.049 | 2.447 | 0.475 |
| 30 | 2.192 | 2.579 | 1.741 | 2.173 | 2.171 | 0.342 |
| 37 | 2.564 | 3.526 | 2.049 | 2.605 | 2.686 | 0.614 |
| 44 | 1.731 | 3.053 | 1.667 | 2.420 | 2.218 | 0.653 |
| 51 | 2.589 | 2.355 | 1.259 | 2.914 | 2.279 | 0.718 |
| 58 | 2.128 | 1.842 | 1.104 | 2.975 | 2.012 | 0.773 |
| 65 | 1.206 | 1.684 | 0.712 | 2.333 | 1.484 | 0.691 |
| 72 | 0.631 | 1.056 | 0.613 | 1.358 | 0.915 | 0.360 |
| 79 | 0.218 | 0.600 | 0.389 | 0.837 | 0.511 | 0.268 |
| 86 | 0.111 | 0.219 | 0.143 | 0.425 | 0.225 | 0.141 |
| 93 | 0.000 | 0.105 | 0.000 | 0.231 | 0.084 | 0.110 |
| 100 | 0.000 | 0.000 | 0.000 | 0.111 | 0.028 | 0.056 |

### Example 6

### Injectability Test of the Microparticles of Example 1-1 Through Small Needles Suitable for Subcutaneous Injection

A cetain amount of microparticles of example 1-1 is reconstituted in 1 ml of the vehicle composition F (Table 3) and shaken by hand for about 30 seconds to form a homogeneous suspension with a suspension concentration as indicated in Table 5. This suspension is withdrawn into a 1 mL syringe. The syringe is fitted with a needle as indicated in Table 5 and inserted into a piece of pork meat (muscle tissue). Once the needle is completely inserted into the muscle tissue the plunger of the syringe is pressed to expell the suspension through the needle into the muscle tissue. The injectability results are indicated in Table 5.

TABLE 5

| Microparticle in vehicle | Needle size | Needle size | Needle size |
|---|---|---|---|
| suspension conc. (mg | 25G × 1″ | 25G × ⅝″ | 23G × 1″ |
| microparticles/mL vehicle) | (0.5 × 25 mm) | (0.5 × 16 mm) | (0.6 × 25 mm) |
| 180-210 mg/mL | Needle clogging - | Needle clogging - | No needle clogging - |
| Dose: 30 mg + 20% | not injectable | not injectable | injectable |
| overfill | | | |
| 110-125 mg/mL | Needle clogging - | No needle clogging - | No needle clogging - |
| Dose: 20 mg + 20% | not injectable | injectable | injectable |
| overfill | | | |
| 60-70 mg/mL | (not determined) | No needle clogging - | (not determined) |
| Dose: 10 mg + 20% | | injectable | |
| overfill | | | |

### Example 7

### Release Profile In Vivo (Rabbits) After I.M. and S.C. Injection

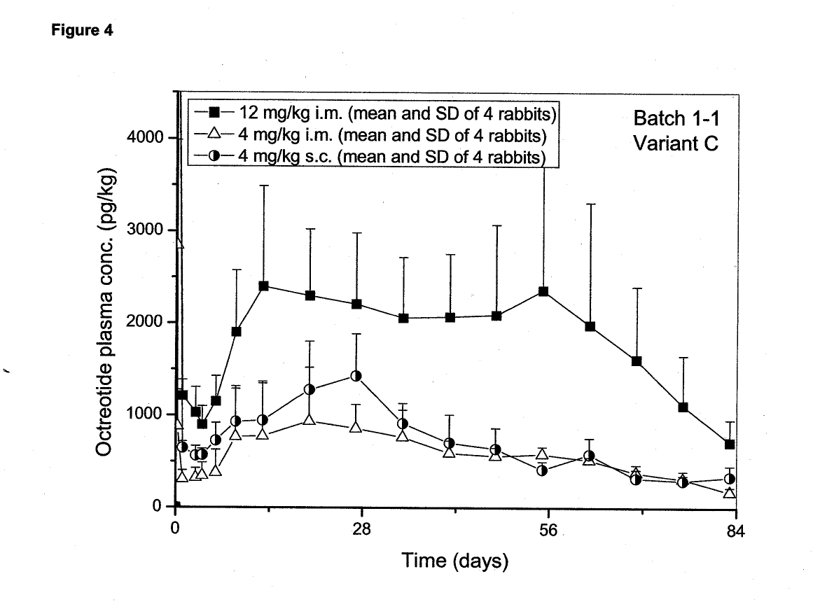

Microparticles of example 1-1 are suspended in 1 ml of a suitable aqueous vehicle and the resulting suspension is injected intramuscularly (i.m.) through a 20 G×1½″ needle at dosages of 4 and 12 mg/kg bw as well as subcutaneously (s.c.) through a 25 G×⅝″ needle at a dosage of 4 mg/kg bw into male New Zealand White rabbits. For each dosage form (test group) 4 animals are used. After defined time periods (indicated as data points in graphs of FIG. 4) plasma samples are taken and analyzed for octreotide concentration by radioimmunoassay (RIA). The resulting release profiles are shown in FIG. 4.

1. A depot formulation comprising as active ingredient octreotide, or a pharmaceutically acceptable salt thereof, and two linear polylactide-co-glycolide polymers (PLGAs) having a molar L:G ratio of 75:25 wherein said polymers have different inherent viscosities between 0.7 dl/g and 0.1 dl/g.

2. A depot formulation according to claim 1 wherein one polymer has an ester and the other polymer has an acid end-group.

3. A depot formulation according to claim 1 wherein the active ingredient is octreotide pamoate.

4. The depot formulation for use according to claim 1 wherein said formulation is administered in dosage strength of 5 to 25 mg.

5. A depot formulation wherein the two polymers are present as polymer blend having a % wt ratio of polymer with higher inherent viscosity to polymer with lower inherent viscosity 85:15 to 50:50.

6. A depot formulation according to claim 1 wherein the viscosities are selected from 0.6 dl/g, 0.4 dl/g or 0.2 dl/g.

7. A depot formulation according to claim 6 wherein said depot formulation is for subcutaneous administration.

8. A depot formulation according to claim 7 wherein the formulation is administered in 0.5 ml to 1.5 ml injection volume.

9. A depot formulation according to claim 1 in form of microparticles, a semisolid or an implant.

10. The depot formulation according to claim 9 in form of microparticles.

11. The depot formulation composition according to claim 10 wherein the microparticles have a diameter between 10 μm and 90 μm.

12. The depot formulation according to claim 9 wherein the microparticles are additionally covered or coated with an anti-agglomerating agent.

13. The depot formulation according to claim 1 sterilized by gamma irradiation.

14. Use of a depot formulation comprising as active ingredient octreotide, or a pharmaceutically acceptable salt thereof, and two different linear polylactide-co-glycolide polymers (PLGAs) having a molar L:G ratio of 85:15 to 65:35 and having two different inherent viscosities of 0.6 dl/g or less for use for the manufacture of a medicament for the treatment of a disease that can be treated by somatostatin analogues, wherein said formulation is administered subcutaneously about monthly in an injection volume of 0.5 ml to 1.5 ml.

15. Use of a pharmaceutical composition according to claim 14 wherein the depot formulation is used in the treatment of severe diarrhea and flushing associated with malignant carcinoid tumors and vasoactive intestinal peptide tumors (vipoma tumors).

16. Use of pharmaceutical composition according to claim 14 wherein the depot formulation is administered at a dosage strength of 5 mg to 25 mg.

17. A method of administering octreotide or a pharmaceutically-acceptable salt thereof for long-term maintenance therapy in acromegalic patients, and treatment of severe diarrhea and flushing associated with malignant carcinoid tumors and vasoactive intestinal peptide tumors (vipoma tumors), said method comprising subcutaneously administering to a patient in need of octreotide, or a pharmaceutically-acceptable salt thereof, as a depot formulation comprising two different linear polylactide-co-glycolide polymers (PLGAs) having a molar L:G ratio of 85:15 to 65:35 and having two different inherent viscosities of 0.6 dl/g or less.

18. A method according to claim 17 wherein the inherent viscosities of the polymers differ 0.2 dl/g to 0.4 dl/g.

19. A process of manufacturing microparticles according to claim 10 comprising
(i) preparation of an internal organic phase comprising
(ia) dissolving the polymers in a suitable organic solvent or solvent mixture;
(ib) dissolving/suspending/emulsification of the drug substance in the polymer solution obtained in step (ia);
(ii) preparation of an external aqueous phase containing stabilizers;
(iii) mixing the internal organic phase with the external aqueous phase to form an emulsion; and
(iv) hardening the microparticles by solvent evaporation or solvent extraction, washing the microparticles, drying the microparticles and sieving the microparticles through 140 μm.

20. An administration kit comprising the pharmaceutical composition according to claim 1 in a vial, together with a water-based vehicle in an ampoule, vial or prefilled syringe or as microparticles and vehicle separated in a double chamber syringe.
